(19) United States
(12) Patent Application Publication
(10) Pub. No.: US 2015/0165141 A1
(43) Pub. Date: Jun. 18, 2015

(54) MEASURING DEVICE OF THE FLOW RATE OF A GASEOUS FLOW FOR MEDICAL IMAGING

(71) Applicant: ISP SYSTEM, Vic-en-Bigorre (FR)

(72) Inventors: Paul Sauvageot, (US); David Brescon, Vic-en-Bigorre (FR); Jean-Marc Cazalet, Vic-en-Bigorre (FR)

(21) Appl. No.: 14/412,927

(22) PCT Filed: Jun. 26, 2013
(86) PCT No.: PCT/FR2013/000162

§ 371 Date: Jan. 5, 2015

(30) Foreign Application Priority Data
Jul. 5, 2012 (FR) 12/01901

(51) Int. Cl. A61M 16/00; A61B 5/087; A61M 16/10; A61B 5/091; A61B 6/03; A61B 5/055

(52) U.S. Cl. CPC A61M 16/0003; A61B 6/037; A61B 5/055; A61B 6/032; A61M 16/0057; A61M 16/1075; A61B 5/091; A61B 5/087; A61M 2016/0027; A61M 2205/3673

(57) ABSTRACT

The invention relates to a measuring device to generate a differential pressure representative of the flow rate of gaseous flow for medical imaging, including: a central body provided with longitudinal channels communicating with an inlet and outlet for the gaseous flow, a casing surrounding the central body, and seals delimiting a first space for measuring an upstream pressure and a second space for measuring a downstream pressure, each of which communicates with at least one of the longitudinal channels, and a third space for warming the central body. The invention also relates to a device to detect the position of a moving area, analysed by medical imaging, of the body of a patient, as well as medical imaging apparatus incorporating such a detection device.

The technical scope of the present invention is that of apparatus to measure gaseous flows. The present invention namely relates to the application of gaseous flow measurement to the medical domain.

The Fleisch cell principle is known for the measurement of a gaseous flow using a differential pressure between an upstream pressure and a downstream pressure. One drawback of devices forming a Fleisch cell, used in medical apparatus, lies in that they are particularly costly given the fact that for reasons of hygiene they must generally be discarded after being used only once.

Another drawback of devices forming a Fleisch cell lies in that they can generally not be used in medical analysis areas subjected to strict safety standards because of the fact that they comprise an electrical heating element arranged in the vicinity of the duct via which the patient breathes. On the one hand, a medical examination may require the absence of electromagnetic waves or the absence of electrical currents or else the absence of dense metallic masses in the analysis area. On the other hand, safety standards generally impose restrictions and stringent conditions of electrical insulation related to the circulation of an electrical current in the vicinity of an element in contact with the patient.

The aim of the present invention is to overcome one or several of the drawbacks to prior art whilst supplying a new device for the measurement of a differential pressure representative of the flow rate of a gaseous flow.

The invention thus relates to a measuring device to generate a representative differential pressure of the flow rate of a gaseous flow for medical imaging, wherein it comprises, at least:
a central body comprising longitudinal channels communicating with an inlet for the gaseous flow and an outlet for the gaseous flow,
a casing surrounding the central body, and
seals arranged between the casing and the central body so as to delimit a first space for measuring an upstream pressure and a second space for measuring a downstream pressure, each of which communicates with at least one longitudinal channel, and a third space supplied with a fluid at a regulated temperature to warm the central body.

According to one characteristic of the invention, the central body and the casing can be disassembled from one another and fit into one another by their longitudinal axis.

According to another characteristic of the invention, the central body comprises peripheral grooves delimiting housings for the seals and delimiting the first, second and third spaces.

According to another characteristic of the invention, the central body comprises heat transfer fins protruding into the third space.

According to another characteristic of the invention, the measuring device comprises four ring-shaped seals successively delimiting between each other the first space, the third space and the second space.

According to another characteristic of the invention, the measuring device comprises an electric air heating system arranged in an air duct to the exterior of the casing and communicating with the third space.

Another subject of the invention is that of a device to detect the position of at least one moving area, analysed by medical imaging, of the body of a patient, wherein it comprises a measuring device and an electronic management module arranged so as to supply data representative of the measurement of the differential pressure between the upstream pressure and the downstream pressure, this data being processed so as to generate a digital signal of data representative of a respiratory volume of the patient in line with an incoming or outgoing digital synchronization signal.

According to another characteristic of the invention, the detection device comprises a portable case to house the measuring device and the electronic management module, the case being offset with respect to the area being analysed. All the electrical parts of the detection device can thus be housed in the case.

According to another characteristic of the invention, the detection device comprises an inhalator through which the patient breathes, this inhalator being joined to a linking tube with a connector at the inlet to the measuring device whose outlet opens into a space in the open air, the connector and the measuring device being offset with respect to the area being analysed. The connector and the measuring device are preferably arranged as closely as possible to the breathing source.

Another subject of the invention relates to medical imaging apparatus comprising at least one medical imaging device of the type positron emission tomography (PET), computed tomography acquisition (CT) or magnetic resonance imaging (MRI), wherein it comprises a detection device according to the invention for the detection of the position of at least one moving area of the body of a patient, this area being analysed by the medical imaging device communicating with the detection device.

A first advantage of the invention lies in the fact that the measuring device can be disassembled in order to be sterilized before being reused, should that prove necessary.

Another advantage lies in the fact that the physical parameters of the measuring device producing the differential pressure remain constant during the different measurements, it is not necessary for the device to be recalibrated for each measurement.

Another advantage of the invention lies in the fact that there can be no current leakage in the duct by which the patient breathes, namely because of the fact that the electrical air heating system is offset with respect to the measuring device.

Yet another advantage lies in the fact that the detection device may be used in medical imaging without generating artefacts and attenuations.

Other characteristics, advantages and particulars of the invention will become more apparent from the additional description hereafter of the different embodiments given by way of illustration and with reference to the appended drawings, in which:

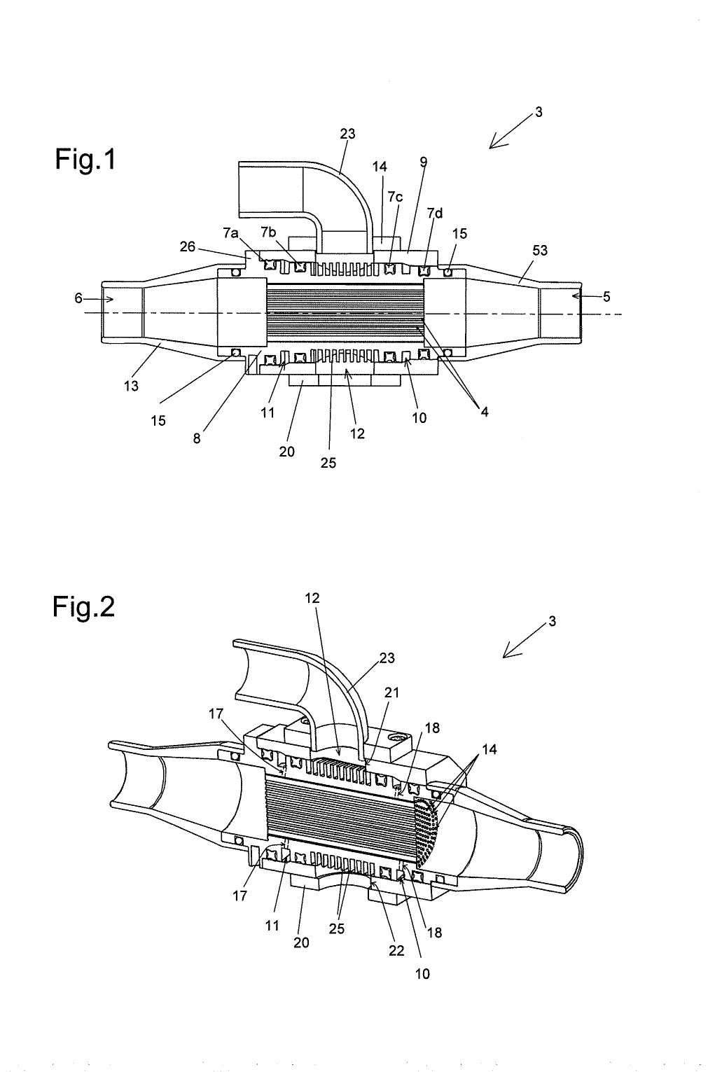

FIG. 1 shows a longitudinal section view of a measuring device generating differential pressures;

FIG. 2 shows a perspective view of the longitudinal section of the device generating differential pressures as shown in FIG. 1;

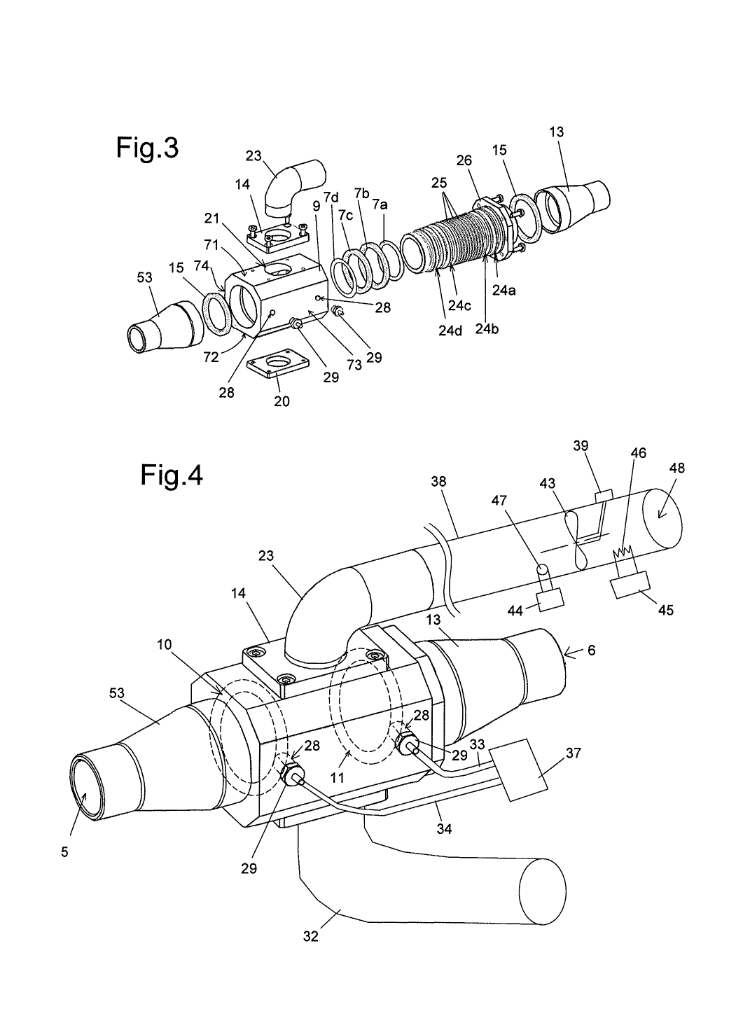

FIG. 3 shown an exploded perspective view of the measuring device generating differential pressures as shown in FIG. 1;

FIG. 4 shows a perspective view of the measuring device generating differential pressures as shown in FIG. 1 connected to the ducts to measure the pressure;

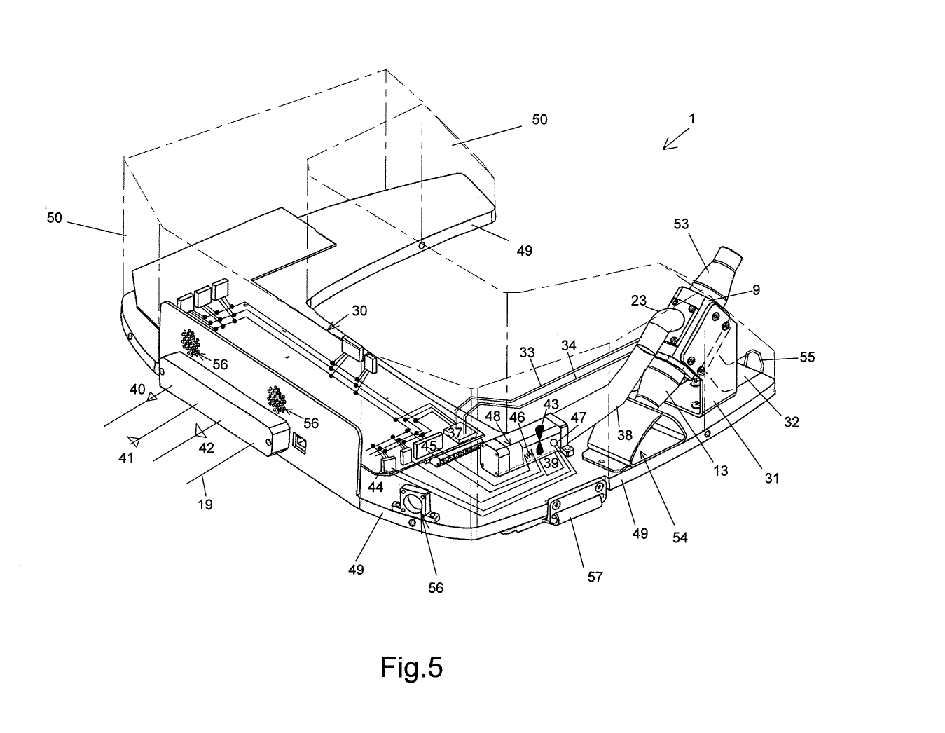

FIG. 5 shows a perspective view of the interior of a device to detect the position of a patient's thorax;

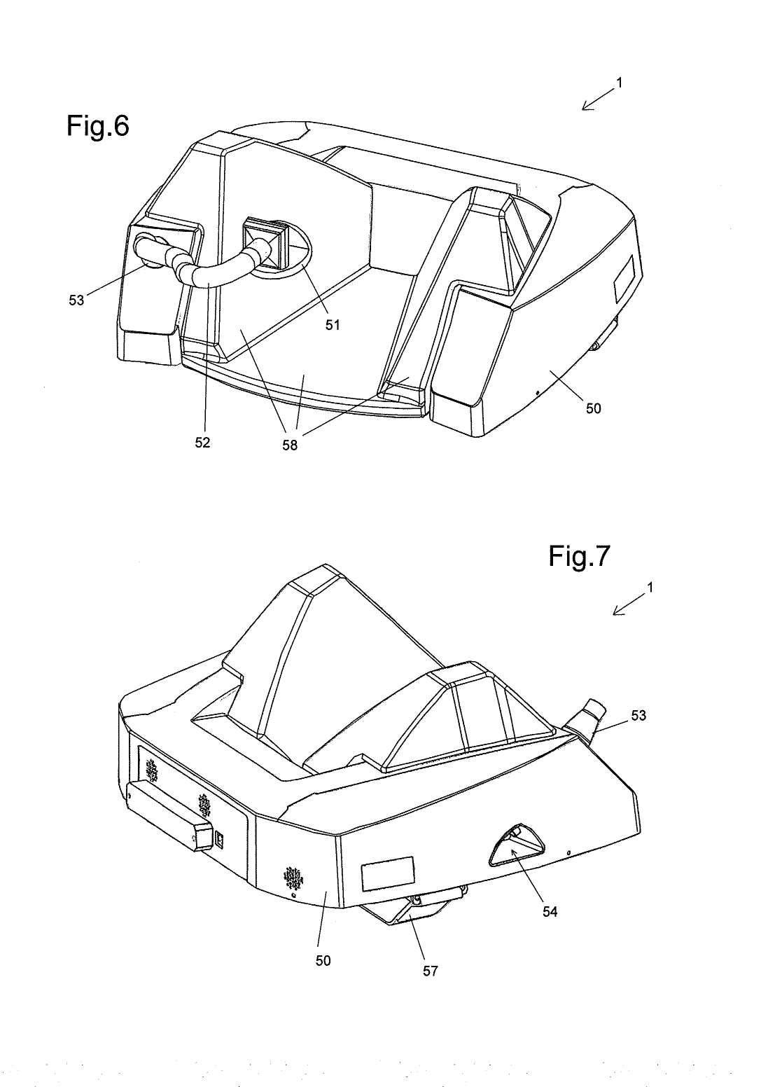

FIG. 6 shows a perspective view of the front of the detection device shown in FIG. 5;

FIG. 7 is a perspective view of the rear of the detection device shown in FIG. 5;

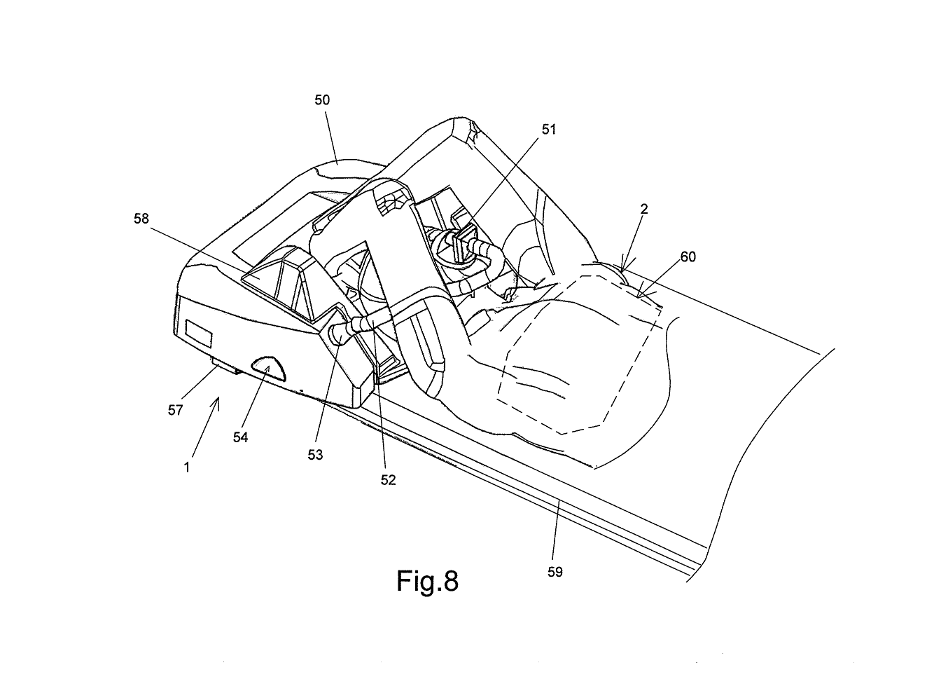

FIG. 8 is a perspective view showing the top of the body of a patient and the detection device in FIG. 5 arranged at the end of a mobile medical table;

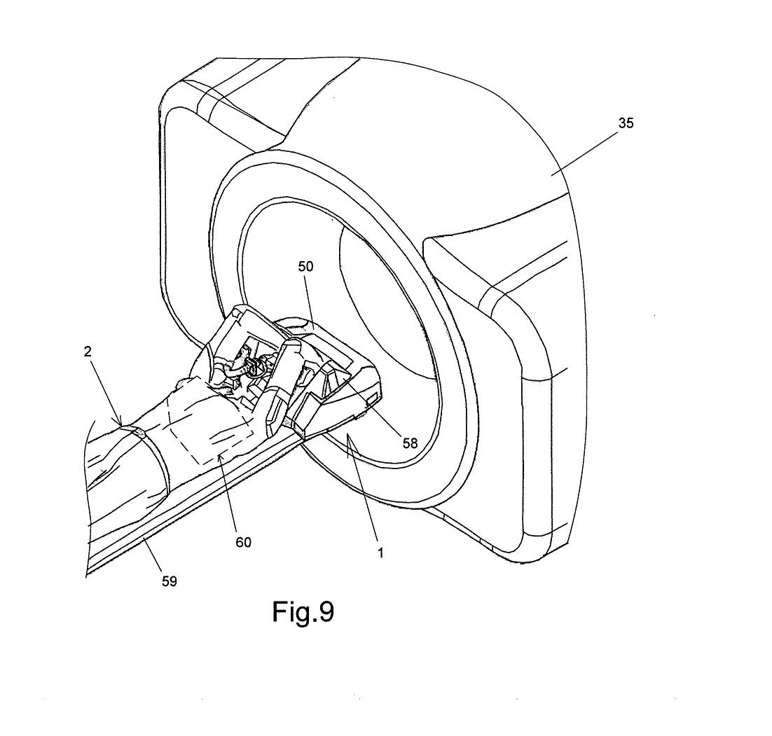

FIG. 9 shows a perspective view of the patient lying on the mobile medical table shown in FIG. 8 and, with the device to detect the position of the patient's body, entering into a medical imaging device;

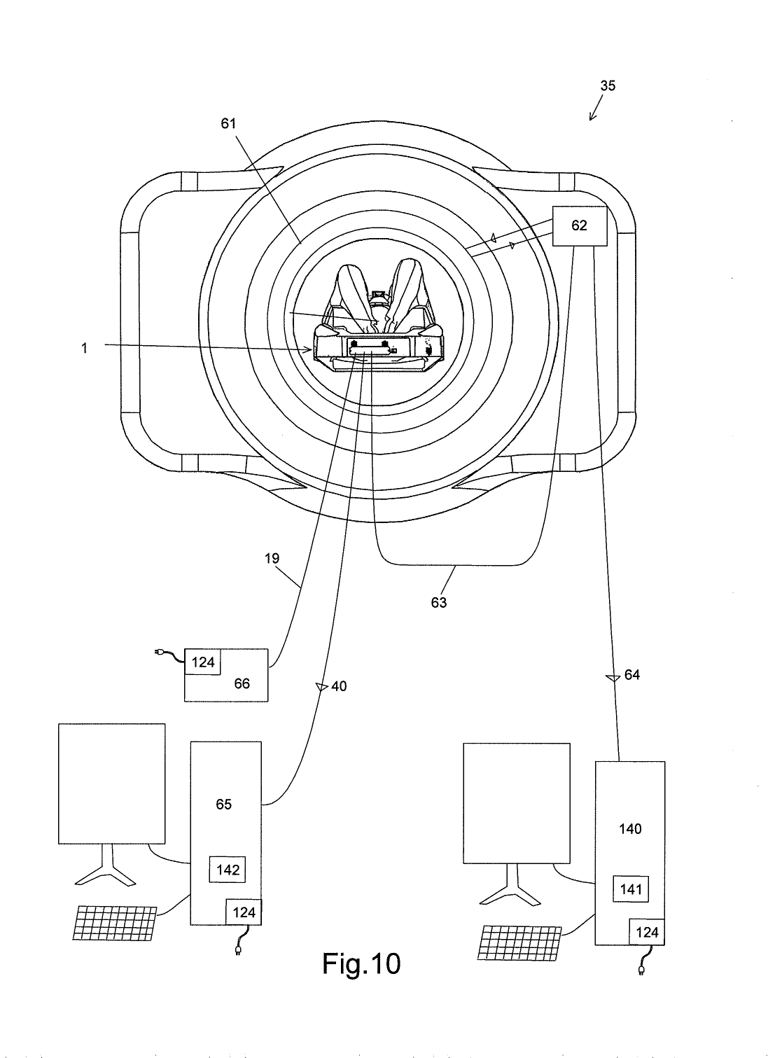

FIG. 10 shows a diagram where a patient is lying on a mobile medical table which is arranged, along with the device to detect the position of the patient's body, in a medical imaging device;

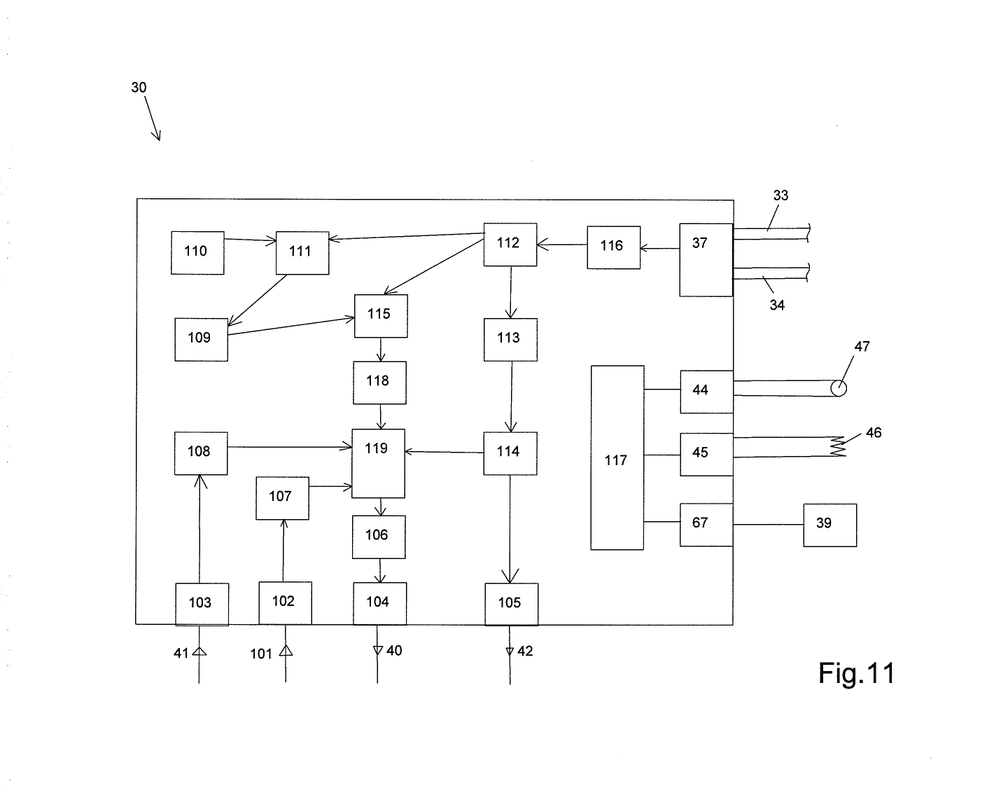

FIG. 11 shows a diagram of a management module for the processing of the data and generation of the output signals; and

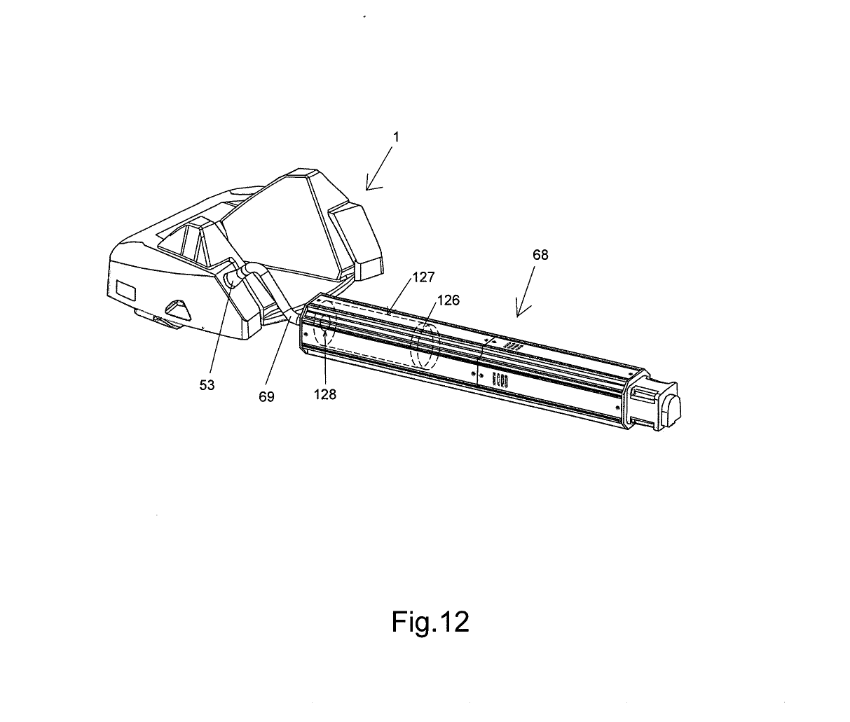

FIG. 12 shows a device to detect the position of a patient's body connected to a calibration device.

The invention will now be described in greater detail. FIG. 1 shows a longitudinal section of a measuring device 3 generating a differential pressure representative of the flow rate of a gaseous flow. This measuring device 3 comprises an inlet 5 and an outlet 6 for the gaseous flow whose flow rate is to be measured.

The terms used to designate the inlet 5 and the outlet for the gaseous flow are not restrictive. Shall be similarly designated the measurements made upstream or respectively downstream performed close to the inlet or respectively outlet. When the patient exhales, the gaseous flow enters by inlet 5 and exits by outlet 6, the flow circulates upstream to downstream.

On the contrary, when the patient inhales, the direction of the gaseous flow is reversed and enters by the outlet before passing via the inlet 5.

The inlet 5 of the gaseous flow is arranged towards the patient and the outlet 6 of the gaseous flow is arranged in a space in the open air. The shapes of the inlet 5 and outlet 6 vents are symmetrical and are tapered and of a length calculated to obtain the same flow rate measurement whether incoming or outgoing.

The measuring device 3 comprises a central body 8 surrounded by a casing 9. The ends of the body protrude at either end of the casing. A hollow connector 53 at the inlet 5 and a hollow connector at the outlet 6 are attached to the ends of the hollow body 8. The hollow body 8 comprises longitudinal channels 4 communicating first with the inlet 5 of the gaseous flow and secondly with the outlet 6 of the gaseous flow. Seals 15 are arranged between the central body 8 and the connectors 53 and 13 at the inlet and outlet. The connectors 13 and 53 are fitted onto the body 8.

Seals 7a, 7b, 7c and 7d arranged between the casing 9 and the central body 8 delimit a first space 10 to measure an upstream pressure and a second space 11 to measure a downstream pressure. The seals 7b and 7c arranged between the casing and the central body 8 also delimit a third space 12 for warming the central body 8, this third space 12 being supplied with fluid at a regulated temperature.

The seals are, for example, O-rings. Any type of ring-shaped seal may be selected, which is to say those with non-circular sections, such as quad-rings.

Four seals 7a, 7b, 7c and 7d successively delimit between each other, the first space 10, the third space 12 and the second space 11.

The casing 9 comprises an inner housing in which the central body 8 is positioned, this inner housing forms several bearings against which a seal is positioned to make air-tight contact. The successive bearings made in the casing 9 are made with decreasing diameters going from one end of the casing abutting against a protruding peripheral shoulder 26 on the central body 8 to the other end of the casing 9 by which the central. body 8 protrudes. The insertion of the central body 8 fitted with its seals is thereby facilitated.

The casing 9 surrounding the central body 8 forms two points of access to the space 12 for warming the central body 8. Plates 14 and 20 attached to the casing 9 comprise an opening in which a duct may be immobilised. These plates 14 and 20 are attached to the casing by screws. In FIG. 1, only one linking duct 23 is attached to the casing 9 by means of a plate 14, the access in the other plate 20 being left free. An evacuation duct linked to this other plate 20 will be described later. The linking duct 23 is intended to be supplied with a fluid at a regulated temperature.

Heat conducting fins 25 are arranged in the central body 8 and protrude in the warming space 12.

As shown in FIG. 2, these fins 25 are in the form of parallel crowns which delimit, between each other, peripheral grooves in the central body 8. In the Figures, the same references are used to designate the same elements.

Warmed air is, for example, injected into the linking duct 23 and then passes through the opening 21 made in the casing to reach the warming space 12. The warm air thus warms the central body 8. The fins 25 enable better distribution of the heat in the central body 8. The warming air injected into the warming space 12 then exits via the opening 22 made in the casing 9. This evacuated warm air is channelled into a vent duct as will be described later. A vent duct is thus attached, in the opening in attachment plate 20 and in communication with the third warming space 12.

Warming the central body 8 makes it possible to avoid the condensation of the air exhaled by the patient which circulates in the central body 8.

The central body 8 comprises a network of parallel channels 4. These longitudinal channels 4 are spaced over the full diameter of the passage for the air flow arranged in the central body.

The air flow passing through these longitudinal channels 4 creates pressure in the longitudinal channels.

Radial ducts 17 and 18 are made in the central body 8 to link one or several longitudinal channels with spaces 10 and 11 to measure the pressures upstream and downstream.

Radial ducts 17 link the external longitudinal channels 4 with the space 11 for measuring the downstream pressure. Radial ducts 18 link the external longitudinal channels 4 with the space 10 for measuring the upstream pressure.

As spaces 10 and 11 for measuring the pressure are closed, the measurement of their internal pressure corresponds to that upstream and downstream in the longitudinal channels. These pressure measurements may thus be used to measure the flow rate of the air flow. Spaces 10 and 11 for measuring the upstream and downstream pressure are delimited by the central body 8 and the casing 9 and, as will be described later, ducts linked to these spaces enable the propagation of their internal pressure. As will be described later, a differential pressure sensor linked to these first and second pressure measuring spaces 10 and 11, make it possible for data representative of the differential pressure to be generated.

Warming the central body 8 as previously described makes it possible to avoid the condensation of the air and the appearance of water droplets which could block the longitudinal channels 4 or the radial ducts 17 and 18 thereby adversely affecting the pressure measurements.

FIG. 3 shows an exploded view of the measuring device.

The exterior of the casing 9 delimits a rectangle parallelepiped comprising longitudinal chamfers. The upper face 71 comprises an opening 21 to access to the warming space 12 and is linked to the attachment plate 14 for the linking duct 23.

The lower face 72 comprises an opening to access to the warming space 12 and is linked to the attachment plate 20 for the linking duct.

The front face 73 as shown in FIG. 3 comprises radial passages 28 opening opposite the upstream and downstream pressure measuring spaces. Connectors 29 are provided to be inserted into these radial passages 28 and attached to this face 73. These connectors 29 are of a shape intended for the connection of two pressure propagation ducts leading to a differential pressure measuring sensor.

The rear face 74 as shown in FIG. 3 is fitted, for example, with threaded holes for the attachment of a support stand for the measuring device.

Housings 24a, 24b, 24c and 24d for seals 7a, 7b, 7c and 7d are in the form of external peripheral grooves. The central body 8 also comprises housings, in the form of external peripheral grooves, delimiting the pressure measuring spaces. External peripheral grooves made in the central body 8 further delimit the cooling fins 25.

The seals 7a, 7b, 7c and 7d are mounted in their housings 24a, 24b, 24c and 24d of the central body 8, then this is inserted in the casing 9 until the shoulder 26 buts against the casing 9. The central body 8 and the casing 9 fit into one another along their longitudinal axis. The casing 9 and the shoulder 26 are attached by screws.

Seals 15 are arranged in housings made in the end collars on which the inlet and outlet connectors 13 and 53 are fitted.

The different elements constituting the measuring device can thus be disassembled, namely to be sterilized. In particular, the central body 8 and the inlet and outlet connectors 13 and 53 can be sterilized. The seals can be sterilized or replaced.

FIG. 4 shows a perspective view of the measuring device to which a duct 38 supplying air at a regulated temperature, a vent duct 32 and pressure propagation ducts 33 and 34 are connected. The ring-shaped spaces 10 and 11 for measuring the differential pressures have been shown in dotted lines. The connectors 29 are arranged in the passages 28 communicating with spaces 10 and 11 for measuring the upstream and downstream pressures. These connectors 29 are also connected to a downstream pressure propagation duct 33 and to an upstream pressure propagation duct 34.

The two pressure propagation ducts 33 and 34 are linked to a differential pressure sensor 37 that is offset with respect to the measuring device 3. The pressure propagation ducts 33 and 34 are, for example, of a length of a few centimetres to a few tens of centimetres. The differential pressure sensor 37 closes each of the pressure propagation ducts 33 and 34 and comprises equipment to supply data representative of the differential pressure. The differential pressure sensor 37 thus supplies data representative of the difference in pressure between the upstream pressure and the downstream pressure in the measuring device. This data is given, for example, in the form of an analogue voltage or in the form of encoded digital data.

To warm the central body, a system of pulsed hot air enables the warming of the central body, the air then being evacuation via a vent duct 32.

The hot pulsed air system, offset with respect to the measuring device, comprises an electrical air heating resistance 46 arranged in a hot air supply duct 38. The heating resistance 46 is powered by a heating module 45. This heating module 45 may be controlled by a management module.

The hot air supply duct 38 is, for example, made of a material that is not electrically conductive so as to avoid any risk of current leakage. This duct 38 is connected to the linking duct 23 communicating with the warming space 12. The air penetrates into the hot air supply duct 38 by an air intake 48. Advantageously, no current circulates in the vicinity of the duct through which the patient breathes and in which the respiratory flow is to be measured.

The air entering by the air intake 48 is driven by a ventilator 43 set into movement by an actuator 39. The actuator 39 may itself be controlled by a management module or can be started as soon as the detection device in which the measuring device is installed is switched on.

A temperature sensor 47 is arranged in the hot air supply duct 38 and is linked to a temperature regulation module 44. This regulation module 44, for example, supplies the management module with data representative of the temperature of the air directed towards the central body. Regulating the heating air temperature thereby makes it possible to avoid the overheating of the central body thereby avoiding overheating the air inhaled by the patient.

The management module controls, for example, the heating module 45 to turn off the heating when the regulation module 44 supplies data representative of the exceeding of a safety threshold temperature stored in the memory of the management module.

Turning off the heating may also be controlled by a bimetallic strip acting as a temperature sensor for the air temperature and mounted in series in the electrical power supply circuit of the electrical heating resistance. A bimetallic strip may also be provided that is attached to one face of the casing 9 or in the heating air vent duct. The short-circuit or open-circuit state of the bimetallic strip can also be controlled by the management module.

After the heated air has circulated between the fins of the central body to warm it, the heating air is evacuated from the casing 9 via a vent duct 32. The vent duct 32 namely enables the heating air to be directed out of an external protective case cover shell.

FIG. 5 shows a detection device 1 for the position of at least one moving area, analysed by medical imaging, of a patient's body where the external shell 50 of the case is shown transparently.

The shell 50 is attached to a base plate 49 to form a housing case. Openings are arranged in the case and namely an opening 55 to expel hot air via the vent duct 32, openings 56 for the intake of air into the inside of the case and an opening into the open air of a space 54 through which the patient breathes.

The support plate 49 is U-shaped, the patient placing his or her head between the branches of the U. The shell 50 extends above the support plate 49. A tether strap 57 for the detection device 1 is attached to the edge of the support plate 49 and passes under the lower face of the support plate 49. The strap 57 makes it possible to attach the device to a mobile medical table, for example. The detection device is advantageously portable.

The case houses the measuring device whose inlet connector 53 protrudes with respect to the shell 50 of the case. A tube may thus be connected by which the patient breathes. The outlet connector 13 communicates with the space 54 in the open air, by which the patient breathes.

The measuring device is attached to a base 31 which is itself attached to the support plate 49. Removing the shell 50 provides access to the measuring device, which may then be removed, namely for sterilization.

The pressure propagation ducts 33 and 34 are fully housed in the case, as is the hot air supply duct 38. The air intake opening 48 is arranged inside the case. When the hot air is directed towards the central body of the measuring device, the air outside the shell penetrates into the shell via aeration openings 56 and is then drawn into the air intake opening 48. The air is made to move namely be the ventilator 43 activated in the hot air supply duct 38. The air is heated by the resistance 46 and regulated by the temperature sensor 47.

After having circulated around the central body, the hot air is evacuated, by the vent duct 32, outside the case.

The case comprises an electronic management module 30 arranged so as to supply data representative of the differential pressure measurement made between the upstream pressure and the downstream pressure, this data being processed to generate a digital data signal 40 representative of a respiratory volume of the patient in line with an incoming 41 or outgoing 42 digital synchronization signal.

The management module 30 comprises, for example, at least one printed circuit. The management module 30 comprises, for example, a data bus, an address bus and a control bus linking together the processing components, the memorization components and the interface components. The memory components are, for example, volatile or non-volatile memories. The processing components are, for example, of the FPGA (Field Programmable Gate Array), DSP (Digital Signal Processor) or ASIC (Application Specific Integrated Circuit) type. The electrical signals are, for example, of the TTL or CMOS type. Shall be designated by module, such as the management module or heating module, a functional assembly comprising a programme or sub-programme memorized and performed to process data or produce data and able to use a working memory space.

The detection device 1 is linked to an electric power cable 19.

The detection device 1 is linked to a communication link supplying an outgoing digital synchronization signal 42. This synchronization signal 42 is produced by the management module 30 using the data representative of the measured flow rate of the air flow.

The detection device 1 is linked to a communication link receiving an incoming digital synchronization signal 41.

The detection device 1 is linked to a communication link and supplies, on this line, an outgoing digital signal 40 representative of the respiratory volume of the patient, in line with a synchronization signal. This synchronization signal is the incoming or outgoing synchronization signal.

For the generation of this signal 40, the management module generates data representative of the respiratory volume of the patient using data representative of the measure flow rate of the air flow.

One example of the processing of the data produced, namely by the differential pressure sensor 37, will be described later with reference to FIG. 11.

FIGS. 6 and 7 show perspective views of the front and rear of the detection device. A set of foam wedge cushions 58 is provided to support the patient's head. The set 58 of foam wedges comprises a lower portion extended by two lateral portions matching the shape of the shell 50 of the detection device. These two lateral portions are fitted against the portions of the shell 50 forming the branches of the U.

The inlet connector 53 protrudes from the shell 50, the other elements constituting the measuring device being arranged in the shell 50. The shell 50 also covers the electronic management module.

The inhalator 51 is linked to the inlet connector via a linking tube 52. The inhalator is, for example, in the form of a mask covering the nose and mouth and comprising an anti-bacterial filter through which the patient breathes. The mask is held in position on the patient's head by an elastic band.

The patient thus breathes through the inhalator linked to the linking tube 52 of the device and the measuring device opening into the open air.

The connector and the measuring device are offset with respect to the area being analysed so as to remain outside this area positioned as closely as possible to the breathing source.

The space 54 in the open air through which the patient breathes is namely shown in FIG. 7.

The portability of the detection device thus enables the measuring device, through which the patient breathes, to be positioned as closely as possible to the patient. Thus, the air circuit through which the patient breathes is of reduced length. The lateral positioning of the measuring device with respect to the patient's head enables the air circuit through which the patient breathes to be further reduced. The reduced length of the circuit makes it possible to have a volume of air that is not entirely renewed but which is tolerable for the patient who breathes through this air circuit for the full duration of the examination.

The lower tether strap 57 passes through buckles attached under the shell.

FIG. 8 shows a detection device attached to an examination table 59 by means of the lower ether strap 57 of the case. The patient's head 2 rests on the foam wedge cushions 58 and between the U-shaped shell 50. The patient lies on the table 59. The table 59 is, for example, able to translate horizontally so as to be inserted into medical imaging apparatus. Throughout the medical examination, the patient breathes through the inhalator 51, the linking tube 52 and the measuring device opening into space 54 in the open air.

An area 60 analysed during the examination has been shown on the patient's 2 rib cage.

Depending on the type of medical imaging device used, shielding for all the electronic parts may be provided. A shielded shell 50 can block the radiation generated during this examination, namely the magnetic radiation used in MRI imaging.

As shown in FIG. 9, the detection device 1 attached to the medical table 59 is moved into the medical imaging device 35 at the same time as the patient 2. The space formed by the shell 50 and foam cushions 58 will be made sufficient for the patient to be able to position his or her head and hands. The positioning of the patient with his or her arms raised and hands locked behind his or her head allows better visualization of the area 60 to be analysed. The U-shape of the detection device in no way hinders the medical imaging process. The inlet connector 53 is namely offset with respect to the patient's head and to the area 60 of the patient to be analysed by medical imaging.

FIG. 10 shows a patient lying on a mobile medical table which is itself arranged along with the detection device 1 for the position of the patient's body, in medical imaging apparatus. This table is associated with a detection device 1 for the position of the moving area of the patient's body 2 to be analysed by medical imaging.

The imaging device comprises a stimulation and detection device 61, schematised by a ring 61, linked to an acquisition and control case 62 for the medical image data representative. The medical image data 64 is transmitted by a communication link to a processing station 140. A storage space 141 is provided for this data which will be analysed later.

The signals transmitted by the medical imaging device and received by the processing station 140 correspond to data representative of medical images in line with the synchronization signal supplied by or transmitted to the detection device.

The communication links between the different stations or devices are coupled by an optical interface enabling an electrical insulation.

The medical imaging device is, for example, of the type positron emission tomography (PET), computed tomography acquisition (CT) or magnetic resonance imaging (MRI).

The medical imaging device is linked by a communication link 63 with the detection device 1 by which a synchronization signal is transmitted. This synchronization signal can be an incoming or outgoing synchronization signal for the detection device 1.

The detection device 1 is linked by its power cable 19 to a power supply unit 66. This power supply unit is connected to the power grid via an isolation transformer 124.

The communication or power supply cables linked to the detection device 1 are selected of a sufficient length to enable the medical table to translate inside the medical imaging device.

The detection device 1 is also linked to its processing station 65 to which it transmits data representative 40 of the respiratory volume in line with the incoming or outgoing synchronization signal. A storage space 142 is provided for this data which will be analysed later.

The processing stations 65 or 140 are, for example, computers equipped with processing programmes and comprising user interfaces. The user interface comprises a screen and a keyboard. The processing stations 140 and 65 are powered by the grid via an isolation transformer 124.

FIG. 11 schematically shows an example of the organisation of the management module 30.

The management module 30 comprises a differential pressure sensor 37 linked to pressure propagation ducts 33 and 34. The differential pressure sensor 37 supplies data representative of the measured differential pressure read by an arithmetic calculation module 116 that supplies data representative of the measured flow rate. The arithmetic calculation module 116 performs, for example, a multiplication of the data representative of a differential pressure to calculate the data representative of a flow rate. The data representative of the measured flow rate are stored in a memory storage space 112.

The memory storage space 112 of the data representative of the measured flow rate is read by an outgoing synchronization signal generation module 113. This module 113, for example, performs comparisons between the successive values ad determines the maximums and minimums of measured flow rate corresponding to synchronization fronts stored in a memory storage space 114 for the outgoing synchronization signal.

The memory storage space 114 for the outgoing synchronization signal is namely read by an interface 105 supplying the outgoing synchronization signal 42.

The memory storage space 112 for the data representative of the measured flow rate is read by a parameterizing module 111 for a respiration model. This parameterizing module 111 can access a memory storage space 110 for a non-parameterized respiration model. The respiration model corresponds to a curve representative of a volume of inhaled and exhaled air by a human being. The non-parameterized model 110 must therefore be parameterized according to each examination. The parameterizing module 111 of the respiration model thus provides access to the data 110 representative of the non-parameterized respiration model and to the data 112 representative of the measured flow rate to generate data 109 representative of the parameterized respiration model, this data being stored in a memory space 109.

The parameterizing module 111 of the respiration model performs an adjustment over a pre-determined number of respiratory cycles. A delay of a few tens of seconds is, for example, planned for the parameterizing of the respiration model. A delay of a few minutes can be planned during which the patient should fall into a regular breathing rhythm.

The parameterizing module 111 of the parameterized respiration model namely comprises a sub-programme to adjust the model's parameters.

Other sub-programmes may be provided in order to parameterize the model such as a self-learning programme to make successive adjustments and error assessments between each adjustment.

The respiration model is a model called a LUJAN model expressed as:

Z(t)=Zo−B.(Cos(π.t/τ(τ−φ)^{2N}

In this function, the position in metres of an organ is given by Z(t).

Zo is an adjustable parameter corresponding to the exhalation position.

B is an adjustable parameter corresponding to the depth of each breath.

Cos is the mathematical function, cosine.

π is the constant of a value of approximately 3.14.

t is the time variable expressed in seconds.

τ is an adjustable parameter corresponding to the period of the respiratory cycle.

φ is an adjustable parameter corresponding to a phase shift.

N is an adjustable parameter corresponding to a degree of asymmetry of the model.

These adjustable parameters are, for example, determined by several samplings and one or several solutions of equation systems.

Determination by equation systems may be combined with self-learning sub-programmes or mean value calculation programmes.

Other respiration models may thus be used.

After the memorizing of the parameterized respiration model 109, a module 115 to generate data representative of the respiratory volume performs a memory access to the parameterized respiration model 109 and to the data 112 representative of the respiration rate. This module 115 generates and writes the data representative of the patient's respiratory volume in a memory space 118.

For the generation of data 118 representative of the respiratory volume, the module 115 which generates it namely comprises a sub-programme for the digital integration of the flow rate.

The management module 30 comprises an interface 103 to receive an incoming synchronization signal 41. The data representative of the incoming synchronization signal is written, by this interface 103, into a memory storage space 108.

The management module 30 comprises an interface 102 to receive at least one command signal 101 for the selection of synchronization with an incoming signal or with an outgoing signal. Other commands may be received to pilot the management module 30. The data representative for this selection command is written, by this interface 102, in a memory storage space 107.

The management module 30 comprises a module 119 to generate the data representative of the patient's respiratory volume in line with an incoming or outgoing synchronization signal, this data being stored in a memory space 106. This memory space 106 is read by an interface 104 generating the outgoing transmission signal 40 for the data representative of the respiratory volume in line with the incoming or outgoing synchronization signal.

Module 119 namely provides access to the data 118 representative of the respiratory volume and to the incoming synchronization data 108 or to the outgoing synchronization data 114 to generate the respiratory volume data 106 in line with the incoming or outgoing synchronization signal. This generation module 119 namely comprises a data concatenation sub-programme. The combination of the respiratory volume data 118 with the incoming synchronization data 108 or with the outgoing synchronization data 114 is made as a function of the state of the memory space 107, accessed by the module 119 to generate the respiratory volume data 106 in line with the incoming or outgoing synchronization signal. The memory space 107 is put into a pre-determined state corresponding to the incoming or outgoing synchronization signal used.

The response time to process a variation of differential pressure translated into data representative of a variation in synchronized respiratory volume with one of the synchronization signals is, for example, less than 12 ms, which can correspond to the normal sampling frequency for a pre-determined pressure differential sensor. The differential pressure sensor is selected according to need. Thus, the management module may be organised so as to have this response time of 15 ms or 30 ms. A real-time system is thus obtained.

The generation of data representative of an activation authorization for the module 119 to generate the data 106 of respiratory volume in line with the incoming or outgoing synchronization signal may also be provided. Such an authorization is, for example, generated by a module 117 to manage the operating temperature.

The module 117 to manage the operating temperature provides read and write access to the working memory spaces of the temperature regulation module 44, the heating module 45 and a control module 67 for the ventilator actuator 39.

The temperature regulation module 117 comprises, for example, a delay sub-programme according to a heating time of the measuring device and a sub-programme to control the heating to a memorized target temperature according to a measured temperature. The module 119 to generate the synchronized respiratory volume data accesses, for example, to an authorization memory space in the temperature management module 117.

FIG. 12 shows a tool 68 to calibrate the detection device 1. A piston 126 driven in translation delimits a chamber 127 comprising an aperture 128 linked by a linking tube 69 to the inlet connector 53 of the detection device 1. By activating the movement of the piston 126 according to the predetermined cycles producing pre-determined air flows, it is possible for the detection device 1 to be calibrated. The calibration tool is, for example, used when a new measuring device is installed or when the device's processing software has been updated or when the processing parameters have been adjusted. Calibration also concerns the synchronization of the spirometer timers and the medical imaging devices as well as that of the Fleisch cells. An inspection may also be performed by way of precaution.

It must be obvious for one skilled in the art that the present invention enables other variant embodiments. Consequently, the present embodiments must be considered as merely illustrative of the invention defined by the attached claims.

1. A measuring device to generate a differential pressure representative of the flow rate of a gaseous flow for medical imaging, wherein it comprises, at least:
a central body comprising longitudinal channels, communicating with an inlet for the gaseous flow and an outlet for the gaseous flow,
a casing surrounding the central body and
seals arranged between the casing and the central body so as to delimit a first space for measuring an upstream pressure and a second space for measuring a downstream pressure, each of which communicates with at least one longitudinal channel, and a third space supplied with a fluid at a regulated temperature for the warming of the central body.

2. A measuring device according to claim 1, wherein the central body and the casing can be disassembled from one another and fit into one another by their longitudinal axis.

3. A measuring device according to claim 1, wherein the central body comprises peripheral grooves delimiting housings for the seals and delimiting the first, second and third spaces.

4. A measuring device according to claim 1, wherein the central comprises heat transfer fins protruding into the third space.

5. A measuring device according to claim 1, wherein it comprises four ring-shaped seals successively delimiting between each other the first space, the third space and the second space.

6. A measuring device according to claim 1, wherein it comprises an electric air heating system arranged in an air duct to the exterior of the casing and communicating with the third space.

7. A device to detect the position of at least one moving area analysed by medical imaging, of the body of a patient, wherein it comprises a measuring device according to claim 1 and wherein it comprises an electronic management module arranged so as to supply data representative of the measurement of the differential pressure between the upstream pressure and the downstream pressure, this data being processed so as to generate a digital signal of data representative of a respiratory volume of the patient in line with an incoming or outgoing digital synchronization signal.

8. A detection device according to claim 7, wherein it comprises a portable case to house the measuring device and the electronic management module, the case being offset with respect to the area being analysed.

9. A detection device according to claim 7, wherein it comprises an inhalator through which the patient breathes, this inhalator being joined to a linking tube with a connector at the inlet to the measuring device whose outlet opens into a space in the open air, the connector and the measuring device being offset with respect to the area being analysed.

10. Medical imaging apparatus comprising at least one medical imaging device of the type positron emission tomography (PET), computed tomography acquisition or magnetic resonance imaging (MRI), wherein it comprises a detection device according to claim 7, for the detection of the position of at least one moving area of the body of a patient, this area being analysed by the medical imaging device communicating with the detection device.
